(19) United States
(12) Patent Application Publication
(10) Pub. No.: US 2010/0056868 A1
(43) Pub. Date: Mar. 4, 2010

(54) ENDOSCOPE BENDING PORTION

(75) Inventor: Hideya Kitagawa, Hachioji-shi (JP)

Correspondence Address: Frishauf, Holtz Goodman & Chick, P.C., 16th Floor, 220 Fifth Avenue, New York, NY, 10001-7708, US

(73) Assignee: Olympus Corporation, Tokyo (JP)

(21) Appl. No.: 12/612,795

(22) Filed: Nov. 5, 2009

Related U.S. Application Data
(63) Continuation of application No. PCT/JP2008/050702, filed on Jan. 21, 2008.

(30) Foreign Application Priority Data
May 31, 2007 (JP) 2007-145630

(51) Int. Cl. A61B 1/008

(52) U.S. Cl. 600/142; 600/141

(57) ABSTRACT

An endoscope bending portion includes bending parts coaxially arranged in a line and each of which includes a cylindrical portion, one bending part of both bending parts adjacent to each other includes a protruding portion integrated with the cylindrical portion and extended in a radial direction of the cylindrical portion, the other bending part of both the bending parts adjacent to each other includes a receiving portion integrated with the cylindrical portion and into which the protruding portion is inserted so as to be rotatable about a longitudinal axis of the protruding portion, and each of the bending parts includes a discontinuous portion extended so as to cross a peripheral direction of the cylindrical portion in the cylindrical portion and joined or unjoined.

### CROSS REFERENCE TO RELATED APPLICATIONS

This is a Continuation Application of PCT Application No. PCT/JP2008/050702, filed Jan. 21, 2008, which was published under PCT Article 21(2) in Japanese.

This application is based upon and claims the benefit of priority from prior Japanese Patent Application No. 2007-145630, filed May 31, 2007, the entire contents of which are incorporated herein by reference.

### BACKGROUND OF THE INVENTION

1. Field of the Invention

The present invention relates to a bending portion provided in an insertion portion of an endoscope and configured to be operated to be bent.

2. Description of the Related Art

An endoscope includes an elongated insertion portion configured to be inserted into a cavity in the body, and a bending portion is provided in the distal end portion of the insertion portion and configured to be operated to be bent. In the bending portion, circular cylindrical bending parts are coaxially coupled to each other so as to be configured to be swung relative to each other. Riveting is used as a method for coupling the bending parts, for example. That is, pairs of tongue portions protrude from both the end surfaces of a circularly cylindrical portion of the bending part, respectively, and the pair of tongue portions is symmetrical about the central axis. The tongue portions of both the bending parts adjacent to each other are overlapped with each other and riveted so as to be configured to be swung relative to each other. Such a method for coupling with riveting is very troublesome, in particular, in a thin endoscope.

In Jpn. Utility Model Appln. KOKOKU Publication No. 61-21042 and Jpn. Pat. Appln. KOKAI Publication No. 2001-104239, a method for coupling without riveting is disclosed. In a bending portion disclosed in Jpn. Utility Model Appln. KOKOKU Publication No. 61-21042, a fitting strip of one bending part of both bending parts adjacent to each other is extended in the axial direction of the bending part and fitted into a locking portion of the other bending part so as to be configured to swung along a contact surface of the bending part. In a bending portion disclosed in Jpn. Pat. Appln. KOKAI Publication No. 2001-104239, coupling strips as tongue portions of both bending parts adjacent to each other are overlapped with each other, and then, a part of the outside coupling strip is made to protrude inward by press from the outside to form a convex portion and, at the same time, fit the convex portion into a through-hole of the inside coupling strip.

### BRIEF SUMMARY OF THE INVENTION

In an aspect of the present invention, an endoscope bending portion includes bending parts coaxially arranged in a line and each of which includes a cylindrical portion, one bending part of both bending parts adjacent to each other includes a protruding portion integrated with the cylindrical portion and extended in a radial direction of the cylindrical portion, the other bending part of both the bending parts adjacent to each other includes a receiving portion integrated with the cylindrical portion and into which the protruding portion is inserted so as to be rotatable about a longitudinal axis of the protruding portion, and each of the bending parts includes a discontinuous portion extended so as to cross a peripheral direction of the cylindrical portion in the cylindrical portion and joined or unjoined.

In another aspect of the present invention, an endoscope includes an endoscope bending portion, the endoscope bending portion includes bending parts coaxially arranged in a line and each of which includes a cylindrical portion, one bending part of both bending parts adjacent to each other includes a protruding portion integrated with the cylindrical portion and extended in a radial direction of the cylindrical portion, the other bending part of both the bending parts adjacent to each other includes a receiving portion integrated with the cylindrical portion and into which the protruding portion is inserted so as to be rotatable about a longitudinal axis of the protruding portion, and each of the bending parts includes a discontinuous portion extended so as to cross a peripheral direction of the cylindrical portion in the cylindrical portion and joined or unjoined.

### BRIEF DESCRIPTION OF THE SEVERAL VIEWS OF THE DRAWING

The accompanying drawings, which are incorporated in and constitute a part of the specification, illustrate embodiments of the invention, and together with the general description given above and the detailed description of the embodiments given below, serve to explain the principles of the invention.

FIG. 1 is a perspective view showing an endoscope according to a first embodiment of the present invention;

FIG. 2 is a perspective view showing a bending tube according to the first embodiment of the present invention;

FIG. 3 is a view for explaining a step of coupling a through-hole bending part in a method for coupling bending parts according to the first embodiment of the present invention;

FIG. 4 is a view for explaining a step of coupling a protruding portion bending part in the method for coupling the bending parts according to the first embodiment of the present invention;

FIG. 5 is a view showing coupled bending parts in the method for coupling the bending parts according to the first embodiment of the present invention;

FIG. 6 is a perspective view showing a bending tube according to a second embodiment of the present invention;

FIG. 7 is a view for explaining a step of coupling a bending part in a method for coupling bending parts according to the second embodiment of the present invention; and

FIG. 8 is a view showing coupled bending parts in the method for coupling the bending parts according to the second embodiment of the present invention.

### DETAILED DESCRIPTION OF THE INVENTION

Hereinafter, each embodiment of the present invention will be explained referring to the drawings.

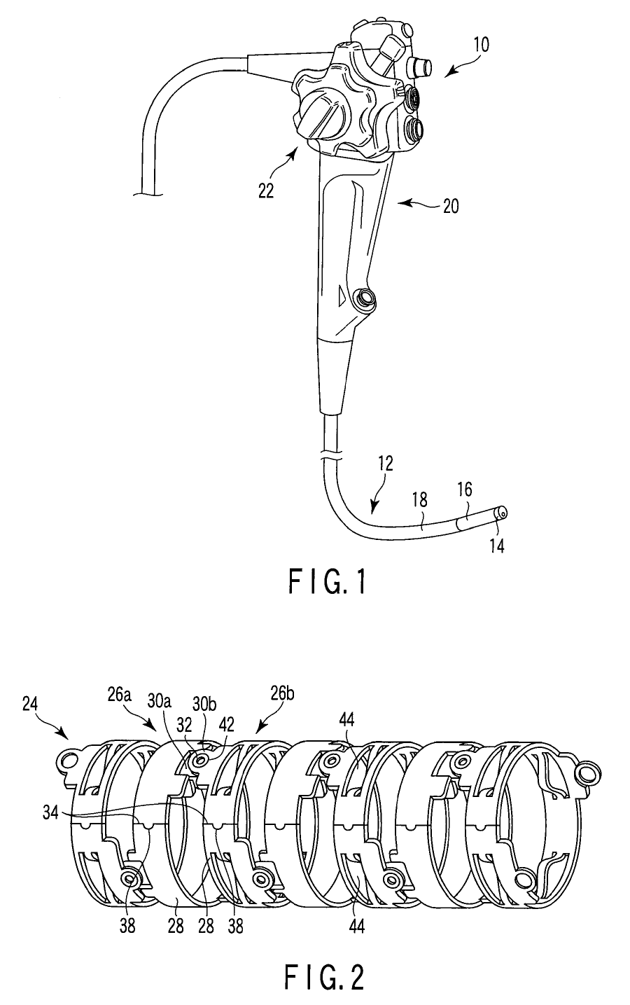

FIGS. 1 to 4 show a first embodiment of the present invention.

Referring to FIG. 1, an endoscope 10 includes an elongated insertion portion 12 configured to be inserted into a cavity in the body. In the insertion portion 12, a distal end rigid portion 14, a bending portion 16 configured to be operated to be bent, a long and flexible insertion tube portion 18 are provided in order from the distal end side. An operating portion 20 is coupled to the proximal end portion of the insertion portion 12 and configured to be grasped and operated. A bending operation knob 22 for operating the bending portion 16 to be bent is provided in the operating portion 20.

Referring to FIG. 2, a tubular bending tube 24 forming a frame of the bending portion 16 will be explained.

In the bending tube 24, two kinds of bending parts, that is, a protruding portion bending part 26a including a protruding portion 32 protruding outward in the radial direction and a through-hole bending part 26b including a through-hole 42 as a receiving portion, are alternately coupled.

In the protruding portion bending part 26a, pairs of protruding portion tongue portions 30a protrude from and are integrated with both the end portions of a circularly cylindrical portion 28 as a cylindrical portion, respectively, and the pair of protruding portion tongue portions 30a is symmetrical about the central axis of the circularly cylindrical portion 28. The pair of protruding portion tongue portions 30a on one end side of the circularly cylindrical portion 28 and that on the other end side thereof are arranged so as to be shifted by 90 degrees relative to each other in the peripheral direction of the circularly cylindrical portion 28. The radial thickness of the protruding portion tongue portion 30a is half as thick as that of the circularly cylindrical portion 28, and the protruding portion tongue portion 30a is arranged on the side close to the inner peripheral surface of the circularly cylindrical portion 28. The protruding portion 32 protrudes from and is integrated with the outer surface of the protruding portion tongue portion 30a. The protruding portion 32 has a shape of a ring and is extended outward in the radial direction, and the height of the protruding portion 32 is half the radial thickness of the circularly cylindrical portion 28. The protruding portion 32 is formed by burring processing, for example. A discontinuous portion 34 is formed in the circularly cylindrical portion 28, and extended orthogonal to the peripheral direction of the circularly cylindrical portion 28, that is, in the axial direction of the circularly cylindrical portion 28. An engaging portion 38 is formed in both the opposite end portions of the circularly cylindrical portion 28 in the discontinuous portion 34 and configured to engage both the end portions with each other. That is, a convex portion is formed in the one end portion of the circularly cylindrical portion 28a and a concave portion is formed in the other end portion, and therefore, the engaging portion 38 wherein the convex portion and the concave portion are engaged with each other is formed. Furthermore, in the discontinuous portion 34, both the end portions of the circularly cylindrical portion 28 are joined to each other by laser welding. That is, the discontinuous portion 34 is joined.

The through-hole bending part 26b includes a circularly cylindrical portion 28, a through-hole tongue portion 30b, a discontinuous portion 34 and an engaging portion 38 similar to those of the protruding portion bending part 26a. Here, the radial thickness of the through-hole tongue portion 30b is half as thick as that of the circularly cylindrical portion 28, and the through-hole tongue portion 30b is arranged on the side close to the outer peripheral surface of the circularly cylindrical portion 28. The through-hole 42 as the receiving portion is radially formed in the through-hole tongue portion 30b. Moreover, wire receivers 44 are formed in and integrated with the circularly cylindrical portion 28 and an operating wire for operating the bending portion 16 to be bent is to be inserted through the wire receiver 44. The wire receiver 44 is formed by making a belt-shaped part, which extends in the peripheral direction in the peripheral wall of the circularly cylindrical portion 28, to protrude inward in the radial direction so as to have a C-shape. The wire receivers 44 are arranged corresponding to the four positions which are shifted relative to each other by 90 degrees and in which the through-hole tongue portions 30b are arranged, respectively, with respect to the peripheral direction of the circularly cylindrical portion 28.

In the bending tube 24, in the protruding portion bending part 26a and the through-hole bending part 26b adjacent to each other, both the tongue portions 30a and 30b are overlapped with each other such that the protruding portion tongue portion 30a is arranged inside and the through-hole tongue portion 30b is arranged outside. The protruding portion 32 of the protruding portion tongue portion 30a is inserted into the through-hole 42 of the through-hole tongue portion 30b so as to be rotatable about the longitudinal axis of the protruding portion 32, and the protruding portion bending part 26a and the through-hole bending part 26b are coupled to each other so as to be configured to be swung relative to each other. Swing directions of bending parts 26a, 26b on the distal end side and the proximal end side of a given bending part 26a, 26b relative to the given bending part 26a, 26b are orthogonal to each other, and the bending tube 24 can be operated to be bent in any direction by combining those swing movements.

It is to be noted that, in the bending tube 24, the discontinuous portions 34 of the protruding portion bending parts 26a and the through-hole bending parts 26b are arranged so as to be aligned with one another in the longitudinally axial direction of the bending tube 24.

Hereinafter, a method for manufacturing the bending portion 16 according to the present embodiment will be explained.

The through-hole bending part 26b and the protruding portion bending part 26a being tubular and including the discontinuous portion 34 which is not joined are formed by press processing. Next, the protruding portion bending part 26a and the through-hole bending part 26b are coupled in order.

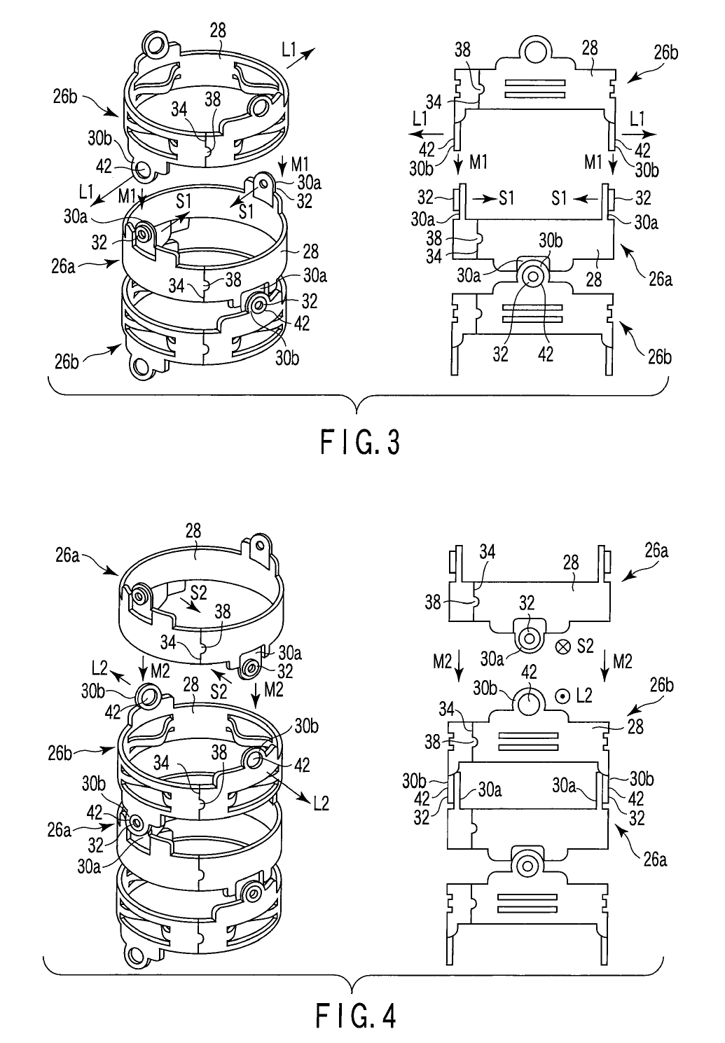

Referring to FIGS. 3 to 5, a method for coupling the protruding portion bending part 26a and the through-hole bending part 26b will be explained.

Referring to FIG. 3, the protruding portion bending part 26a shown on the upper side has been coupled to the through-hole bending part 26b shown on the lower side, and next through-hole bending part 26b shown on upper side will be coupled to the coupled protruding portion bending part 26a. Hereinafter, a step of coupling the next through-hole bending part 26b to the coupled protruding portion bending part 26a will be explained.

The next through-hole bending part 26b is arranged so as to be coaxial with the coupled protruding portion bending part 26a and the through-hole bending part 26b is aligned with the coupled protruding portion bending part 26a with respect to the peripheral direction such that the through-hole tongue portion 30b is opposite to the protruding portion tongue portion 30a. At this time, the discontinuous portion 34 of the protruding portion bending part 26a and that of the through-hole bending part 26b are aligned with each other with respect to the peripheral direction. Next, as is shown by arrow L1 in the figure, the through-hole bending part 26b is increased in diameter by increasing the space of the discontinuous portion 34, and the through-hole 42 is moved outward in the radial direction and maintained. On the other hand, in the through-hole bending part 26b and the protruding portion bending part 26a coupled to each other, the through-hole tongue portion 30b is arranged outside and the protruding portion tongue portion 30a is arranged inside, and the protruding portion 32 protrudes outward in the radial direction, and therefore, the coupled protruding portion bending part 26a can be decreased in diameter. As is shown by arrow S1 in the figure, the coupled protruding portion bending part 26a is decreased in diameter by reducing the space of the discontinuous portion 34 and, if necessary, overlapping both the end portions of the circularly cylindrical portion 28 with each other in the discontinuous portion 34 and the protruding portion 32 is moved inward in the radial direction and maintained. The protruding end portion of the protruding portion 32 is arranged so as to be positioned slightly radially inside of the through-hole 42 by the movement of the through-hole 42 outward in the radial direction and the movement of the protruding portion 32 inward in the radial direction. Here, in the through-hole bending part 26b and the protruding portion bending part 26a coupled to each other, although the protruding portion bending part 26a is decreased in diameter, the through-hole bending part 26b is not increased in diameter, and therefore, the whole of the protruding portion 32 is not pulled out from the through-hole 42. Next, as is shown by arrow M1 in the figure, the through-hole bending part 26b is moved in the axial direction toward the coupled protruding portion bending part 26a, and the through-hole 42 is positioned radially outside of the protruding portion 32. Then, the decrease in diameter of the protruding portion bending part 26a and the increase in diameter of the through-hole bending part 26b are released, and the protruding portion 32 is moved outward in the radial direction and the through-hole 42 is moved inward in the radial direction, and the protruding portion 32 is inserted into the through-hole 42. At this time, in the discontinuous portion 34, the convex portion and the concave portion of both the end portions of the circularly cylindrical portion 28 are engaged with each other, and both the end portions are positioned relative to each other with respect to the axial direction.

In this way, as is shown in the lower side of FIG. 4, the through-hole bending part 26b is coupled to the protruding portion bending part 26a.

Referring to FIG. 4, a step of coupling next protruding portion bending part 26a to the coupled through-hole bending part 26b will be explained further.

The step of coupling the next protruding portion bending part 26a to the coupled through-hole bending part 26b is similar to the above mentioned step of coupling the next through-hole bending part 26b to the coupled protruding portion bending part 26a. However, as is shown by arrow L2 in the figure, the coupled through-hole bending part 26b is increased in diameter to move the through-hole 42 outward in the radial direction, and, as is shown by arrow S2 in the figure, the next protruding portion bending part 26a is decreased in diameter to move the protruding portion 32 inward in the radial direction. Here, in the protruding portion bending part 26a and the through-hole bending part 26b coupled to each other, the protruding portion tongue portion 30a is arranged inside, the protruding portion 32 protrudes outward in the radial direction, and the through-hole tongue portion 30b is arranged outside, and therefore, the coupled through-hole bending part 26b can be increased in diameter.

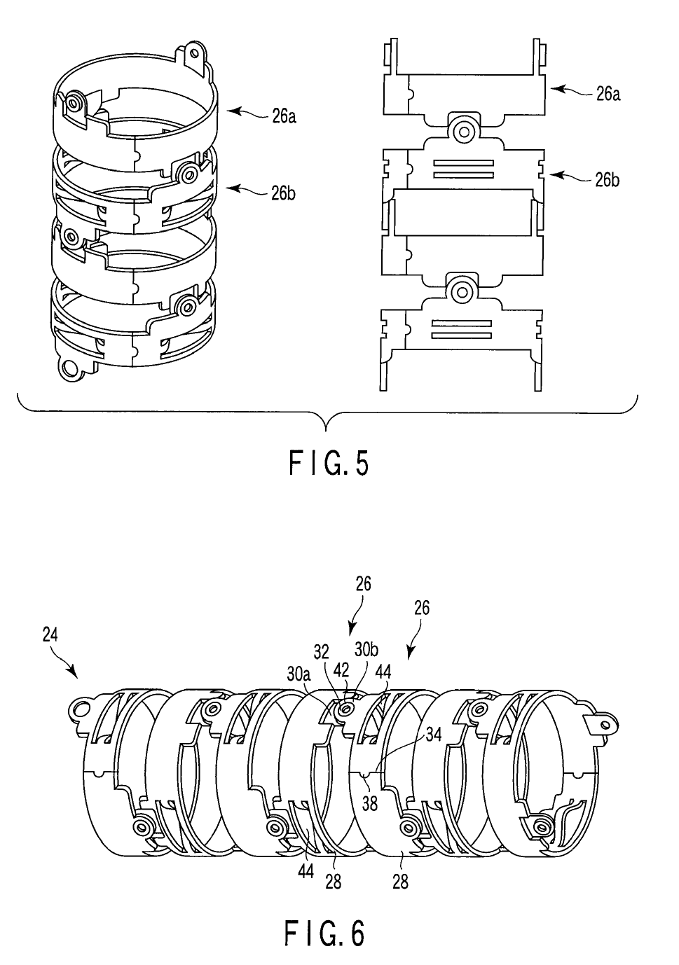

In this way, as is shown in FIG. 5, the next protruding portion bending part 26a is coupled to the through-hole bending part 26b.

As is similar to the above, the protruding portion bending part 26a and the through-hole bending part 26b are coupled in order. After coupling, in the bending tube 24, the discontinuous portions 34 of the protruding portion bending parts 26a and the through-hole bending parts 26b are arranged so as to be aligned with one another in the longitudinally axial direction of the bending tube 24. Moreover, both the end portions of the circularly cylindrical portion 28 are positioned relative to each other with respect to the axial direction in the discontinuous portion 34. Next, the discontinuous portions 34 of the protruding portion bending parts 26a and the through-hole bending parts 26b are joined in order by laser welding.

The bending portion 16 according to the present embodiment exhibits following effects.

In the bending portion 16 according to the present embodiment, the protruding portion 32 extended in the radial direction of the protruding portion bending part 26a is inserted into the through-hole 42 of the through-hole bending part 26b so as to be rotatable about the longitudinal axis of the protruding portion 32. Moreover, the tubular protruding portion bending part 26a and the tubular through-hole bending part 26b are precisely formed by press processing, and then, the protruding portion 32 and the through-hole 42 are displaced in the radial direction relative to each other by a decrease in diameter of the protruding portion bending part 26a and an increase in diameter of the through-hole bending part 26b using the discontinuous portion 34, and the protruding portion 32 and the through-hole 42 are positioned relative to each other with respect to the axial direction and the peripheral direction by a relative movement of the protruding portion bending part 26a and the through-hole bending part 26b, and, after that, the protruding portion 32 is inserted into the through-hole 42 by a release of the decrease in diameter of the protruding portion bending part 26a and the increase in diameter of the through-hole bending part 26b to move the protruding portion 32 and the through-hole 42 in the radial direction relative to each other, whereby the bending portion 16 is manufactured. Therefore, it is possible to easily manufacture the bending portion 16 which can be smoothly operated to be bent.

Moreover, the through-hole bending part 26b configured to be increased in diameter and the protruding portion bending part 26a configured to be decreased in diameter are alternately coupled, and, when next through-hole bending part 26b or next protruding portion bending part 26a is coupled to the coupled protruding portion bending part 26a or the coupled through-hole bending part 26b, the coupled protruding portion bending part 26a or the through-hole bending part 26b as well as the next through-hole bending part 26b or the next protruding portion bending part 26a can be increased or decreased in diameter. Therefore, it is possible to restrict amount of deformation of the protruding portion bending part 26a and the through-hole bending part 26b in the coupling and prevent strain of the protruding portion bending part 26a and the through-hole bending part 26b due to the deformation.

Furthermore, the convex portion and the concave portion of both the end portions of the circularly cylindrical portion 28 are engaged with each other in the discontinuous portion 34 and both the end portions of the circularly cylindrical portion 28 are positioned relative to each other with respect to the axial direction. Therefore, an apparatus and a step for positioning both the end portions of the circularly cylindrical portion 28 in laser welding is unnecessary, and it is possible to easily and inexpensively perform the laser welding.

In addition, the discontinuous portions 34 of the bending parts 26a and 26b are aligned with one another in the longitudinally axial direction of the bending tube 24. Therefore, places to be welded by the laser welding are aligned with one another in the longitudinally axial direction of the bending tube 24, and it is possible to easily perform the welding step.

It is to be noted that a protruding portion may protrude from the inner surface of a tongue portion and inward in the radial direction in a protruding portion bending part, the protruding portion tongue portion may be arranged outside, a through-hole tongue portion may be arranged inside. In this case, in a step of coupling the protruding portion bending part and a through-hole bending part, the protruding portion bending part is increased in diameter and the through-hole bending part is decreased in diameter.

FIGS. 6 to 8 show a second embodiment of the present invention.

Referring to FIG. 6, in a bending tube 24 according to the present embodiment, one kind of bending parts 26 are coupled in order, a protruding portion 32 is provided on the one end side of the bending part and a through-hole 42 as a receiving portion is provided on the other end side thereof.

The bending part 26 includes a circularly cylindrical portion 28, a protruding portion tongue portion 30a, a through-hole tongue portion 30b, a discontinuous portion 34, an engaging portion 38 and a wire receiver 44 similar to those of the protruding portion bending part 26a or the through-hole bending part 26b according to the first embodiment, respectively.

That is, in the bending part 26, a pair of protruding portion tongue portions 30a is provided on one end side of the circularly cylindrical portion 28 and a pair of through-hole tongue portion 30b is provided on the other end side thereof. The pair of protruding portion tongue portion 30a and the pair of through-hole tongue portion 30b are arranged so as to be shifted by 90 degrees relative to each other in the peripheral direction of the circularly cylindrical portion 28. The wire receivers 44 are arranged corresponding to the two positions in which the through-hole tongue portions 30b are arranged, respectively, with respect to the peripheral direction of the circularly cylindrical portion 28.

In the bending tube 24, as is similar to the first embodiment, the protruding portion 32 of the protruding portion tongue portion 30a and the through-hole 42 of the through-hole tongue portion 30b are coupled to each other in both the bending parts 26 adjacent to each other. In both the bending parts 26 adjacent to each other, the pair of wire receivers 44 on the one bending part 26 and the pair of wire receivers 44 on the other bending part 26 are arranged so as to be shifted by 90 degrees relative to each other in the peripheral direction of the bending tube 24. That is, in the bending tube 24, the wire receivers 44 are arranged in the four positions with respect to the peripheral direction of the circularly cylindrical portion 28. The discontinuous portions 34 of the bending parts 26 are arranged so as to be shifted by 90 degrees in order and circulated in the peripheral direction of the central axis of the bending tube 24. The discontinuous portion 34 is not joined and kept unjoined.

Hereinafter, a method for manufacturing the bending portion 16 according to the present embodiment will be explained.

One kind of bending parts 26 being tubular and each of which includes the discontinuous portion 34 which is not joined is formed by press processing. Next, the bending parts 26 are coupled in order.

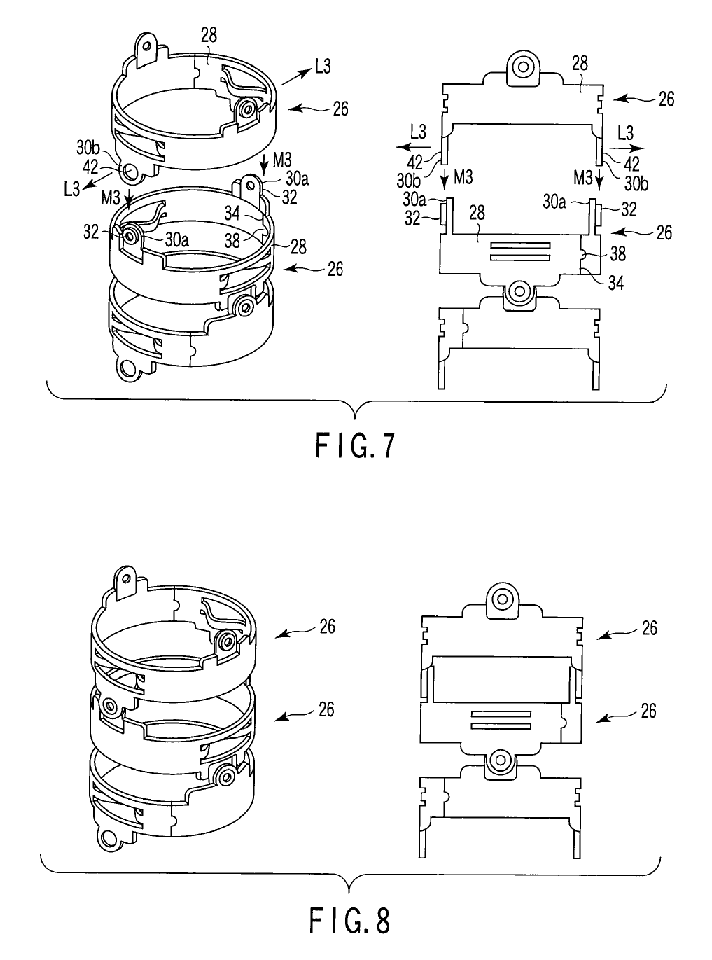

Referring to FIGS. 6 and 7, a method for coupling the bending parts 26 will be explained. In the present embodiment, the protruding portion 32 of the coupled bending part 26 is inserted into the through-hole 42 of next bending part 26.

Referring to FIG. 7, the next bending part 26 on the upper side is arranged so as to be coaxial with the coupled bending part 26 on the lower side and the next bending part 26 and the coupled bending part 26 are aligned with each other with respect to the peripheral direction such that the through-hole tongue portion 30b is opposite to the protruding portion tongue portion 30a. At this time, the discontinuous portion 34 of the next bending part 26 is arranged so as to be shifted by 90 degrees in one orientation of the peripheral direction relative to the discontinuous portion 34 of the coupled bending part 26. Next, as is shown by arrow L3 in the figure, the next bending part 26 is increased in diameter by increasing the space of the discontinuous portion 34, and the through-hole 42 is moved outward in the radial direction and maintained. The through-hole 42 is arranged so as to be positioned slightly outside of the protruding end portion of the protruding portion 32 in the radial direction by the movement of the through-hole 42 outward in the radial direction. Next, as is shown by arrow M3 in the figure, the next bending part 26 is moved in the axial direction toward the coupled bending part 26, and the through-hole 42 is positioned radially outside of the protruding portion 32. Then, the increase in diameter of the next bending part 26 is released, the through-hole 42 is moved inward in the radial direction, and the protruding portion 32 is inserted into the through-hole 42. At this time, in the discontinuous portion 34, the convex portion and the concave portion of both the end portions of the circularly cylindrical portion 28 are engaged with each other, and both the end portions are positioned relative to each other with respect to the axial direction and maintained.

In this way, as is shown in FIG. 8, both the bending parts 26 are coupled.

Furthermore, the bending tube 24 is covered with an outer tube, and the bending part 26 in which the discontinuous portion 34 is unjoined is prevented from being increased or decreased in diameter.

The bending portion 16 according to the present embodiment exhibits following effects.

In the bending portion 16 according to the present embodiment, the bending portion 16 is made of the one kind of bending parts 26, and manufacturing cost for the bending part 26 is reduced, and also, the step of coupling the bending part 26 is simplified. Therefore, it is possible to inexpensively manufacture the bending portion 16.

Moreover, the convex portion and the concave portion of both the end portions of the circularly cylindrical portion 28 are engaged with each other in the discontinuous portion 34 and both the end portions of the circularly cylindrical portion 28 are positioned relative to each other with respect to the axial direction and maintained. Therefore, it is possible to secure the axial strength of the bending part 26.

Furthermore, the discontinuous portions 34 of the bending parts 26 are arranged so as to be shifted by 90 degrees in order and circulated in the peripheral direction of the central axis of the bending tube 24. Therefore, the strength of the bending portion 16 is uniform with respect to the peripheral direction and an especially weak place is not formed.

It is to be noted that a protruding portion of next bending part may be inserted into a through-hole of a coupled bending part in a method for coupling a bending part. In this case, in a step of coupling a bending part, the next bending part is decreased in diameter. A protruding portion may protrude from the inside surface of a protruding portion tongue portion and inward in the radial direction, the protruding portion tongue portion may be arranged outside, and a through-hole tongue portion may be arranged inside. In this form, in the case where a protruding portion of a coupled bending part is inserted into a through-hole of a next bending part, the next bending part is decreased in diameter, and, in the case where a protruding portion of a next bending part is inserted into a through-hole of a coupled bending part, the next bending part is increased in diameter.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. An endoscope bending portion comprising bending parts coaxially arranged in a line and each of which includes a cylindrical portion,
wherein one bending part of both bending parts adjacent to each other includes a protruding portion integrated with the cylindrical portion and extended in a radial direction of the cylindrical portion,
the other bending part of both the bending parts adjacent to each other includes a receiving portion integrated with the cylindrical portion and into which the protruding portion is inserted so as to be rotatable about a longitudinal axis of the protruding portion, and
each of the bending parts includes a discontinuous portion extended so as to cross a peripheral direction of the cylindrical portion in the cylindrical portion and joined or unjoined.

2. The endoscope bending portion according to claim 1,
wherein the one bending part includes both the protruding portions provided on both end sides of the cylindrical portion, respectively, and protruding outward or inward in the radial direction, and
the other bending part includes both the receiving portion provided on both end sides of the cylindrical portion.

3. The endoscope bending portion according to claim 1,
wherein each of both the bending parts adjacent to each other includes the protruding portion provided on one end side of the cylindrical portion and the receiving portion provided on the other end side of the cylindrical portion.

4. The endoscope bending portion according to claim 1,
wherein each of the bending parts includes an engaging portion provided in both opposite end portions of the cylindrical portion in the discontinuous portion and engaging both the end portions with each other.

5. The endoscope bending portion according to claim 1,
wherein the discontinuous portions of the bending parts are aligned with each other in a longitudinally axial direction of the bending portion.

6. The endoscope bending portion according to claim 1,
wherein the discontinuous portions of the bending parts are arranged so as to be shifted relative to each other in a peripheral direction of a central axis of the bending portion.

7. An endoscope comprising an endoscope bending portion,
wherein the endoscope bending portion includes bending parts coaxially arranged in a line and each of which includes a cylindrical portion,
one bending part of both bending parts adjacent to each other includes a protruding portion integrated with the cylindrical portion and extended in a radial direction of the cylindrical portion,
the other bending part of both the bending parts adjacent to each other includes a receiving portion integrated with the cylindrical portion and into which the protruding portion is inserted so as to be rotatable about a longitudinal axis of the protruding portion, and
each of the bending parts includes a discontinuous portion extended so as to cross a peripheral direction of the cylindrical portion in the cylindrical portion and joined or unjoined.

8. The endoscope according to claim 7,
wherein the one bending part includes both the protruding portions provided on both end sides of the cylindrical portion, respectively, and protruding outward or inward in the radial direction, and
the other bending part includes both the receiving portion provided on both end sides of the cylindrical portion.

9. The endoscope according to claim 7,
wherein each of both the bending parts adjacent to each other includes the protruding portion provided on one end side of the cylindrical portion and the receiving portion provided on the other end side of the cylindrical portion.

10. The endoscope according to claim 7,
wherein each of the bending parts includes an engaging portion provided in both opposite end portions of the cylindrical portion in the discontinuous portion and engaging both the end portions with each other.

11. The endoscope according to claim 7,
wherein the discontinuous portions of the bending parts are aligned with each other in a longitudinally axial direction of the bending portion.

12. The endoscope according to claim 7,
wherein the discontinuous portions of the bending parts are arranged so as to be shifted relative to each other in a peripheral direction of a central axis of the bending portion.
